(19) United States
(12) Patent Application Publication
(10) Pub. No.: US 2024/0366526 A1
(43) Pub. Date: Nov. 7, 2024

(54) COMPOSITIONS AND METHODS FOR MAINTAINING OR ENHANCING MICROBIAL BALANCE OF THE SKIN AND MUCOSA

(71) Applicant: Reckitt Benckiser Health Limited, Slough (GB)

(72) Inventors: Sagarika Banerjee, Montvale, NJ (US); Jin Seo, Montvale, NJ (US)

(21) Appl. No.: 18/653,077

(22) Filed: May 2, 2024

Related U.S. Application Data
(60) Provisional application No. 63/499,800, filed on May 3, 2023.

(51) Int. Cl. A61K 31/05; A61K 9/00; A61K 31/045; A61P 31/04; A61P 31/10

(52) U.S. Cl. CPC A61K 31/05; A61K 9/0014; A61K 31/045; A61P 31/04; A61P 31/10

(57) ABSTRACT

The present disclosure is related to a composition and method for the treatment and/or prevention of skin and/or mucosal conditions resulting from microbial overgrowth, imbalance, or infections. The method comprises administering a composition to a subject that comprises in particular carvacrol and Terpinen-4-ol in amounts and mutual ratios to maintain a balance between beneficial, commensal microorganisms and non-beneficial, opportunistic pathogenic microorganisms.

### CROSS-REFERENCE TO RELATED APPLICATIONS

This application claims the benefit under 35 USC § 119(e) of U.S. Provisional Application No. 63/499,800, filed May 3, 2023. The entire contents of the above-referenced patent application(s) are hereby expressly incorporated herein by reference.

### TECHNICAL FIELD

The present disclosure is related to a topical composition and method for the treatment and/or prevention of skin, and mucosal conditions resulting from microbial overgrowth, imbalance or infections. More specifically, the topical composition comprises in particular carvacrol and Terpinen-4-ol in amounts and mutual ratios to maintain a balance between beneficial and non-beneficial microorganisms.

### BACKGROUND

The skin is the outermost tissue or organ of the body in most species and is prone to contact with potentially harmful microorganisms whether by direct contact or by airborne or water borne spread.

Many different types of microbial agents may cause infection of the skin or mucosa. These include bacteria, viruses, and fungal species (including yeasts) which may cause symptoms which include irritation, itching, scaling (scaly skin), redness, swelling, blisters, vesicles and other lesions. Common infections can be bacterial, such as staphylococcal infections, viral infections such as shingles (caused by Herpes zoster), Herpes simplex infections and viral warts (by Human Papillomavirus), and fungal infections (mycoses) such as yeast infections (e.g., Candida sp., Malassezia sp.), athlete's foot (Tinea pedis), ringworm (Tinea corporis), seborrheic dermatitis.

Cells lining the lower female reproductive tract, including that of the vulva, vagina and external cervix, together with the resident commensal, vaginotropic microbiome (collectively, the “vaginal ecosystem”), form a dynamic organ that not only protects against infectious disease but also maintains tissue health and facilitates reproduction. The vaginal ecosystem is made up of a variety of factors that together act to support homeostasis of this ecosystem, including immune active cells, a protective mucus coating, a variety of antimicrobial peptides, pH, and symbiotic bacteria (e.g., Lactobacilli).

To maintain homeostasis, the vaginal ecosystem must adapt to changes that occur during a woman's monthly hormone cycle, as well as adjust to more profound changes that happen at different times in the arc of a woman's life, such as puberty, reproductive years, perimenopause and menopause. For example, during various stages of a woman's reproductive cycle, the vaginal ecosystem is continually providing a protective barrier to diseases while at the same time lubricating the vagina to accommodate for sexual intercourse, or facilitating the passage of blood out of the uterus at menses, or inhibiting/optimizing sperm transport through the vagina and cervix, or undergoing chemical changes to promote labor and birth of a baby.

Throughout a woman's life, the lower female reproductive tract and vaginal ecosystem continually experience various disturbances (acute and chronic) caused by human behavior (e.g., sexual intercourse), but also by the use of contraception and vaginal products (Hickey et al, Transl. Res. 160:261, 2012; Ma et al, Annu. Rev. Microbiol. 66:371, 2012). If disturbance of the vaginal ecosystem becomes chronic, then discomfort, and even diseases, can ensue. Any vaginal product that alters the mucosal environment and impairs the epithelial barrier may increase the severity of symptoms or risk of diseases (Fichorova et al., Toxicol. Appl. Pharmacol. 255:198, 2015; Ma et al., 2012).

Several different plant-derived substances have previously been used in an attempt to provide a gentler, natural solution to the problem of skin or mucosal infections associated with the above-mentioned microbial organisms. However, in many cases, although these natural compositions are associated with fewer harmful effects than conventional pharmaceutical agents, they often have unacceptably low levels of efficacy and/or non-targeted.

Terpenes, such as carvacrol and Tea tree oil and Terpinen-4-ol, among others, has been used in combination with other ingredients as an antimicrobial for medical and veterinary purposes for some time (WO 2007/063267, WO 2007/063268). Terpenes have even been used as an antimicrobial in oral uses for plaque and halitosis (BR102019027239 A2, WO12064319 A1, WO07056491 A2). Further, it has been known as a preservative/broader antimicrobial for products that maintain or enhance homeostasis or function of female lower reproductive tract (WO 2017/180174) or in addition with other actives, such as Nigella sativa seeds, to treat microbial overgrowth, imbalance and infections in skin, mucosal and systemic conditions (WO22/254446).

Terpenes have also been used in combinations, that include Carvacrol and Terpinen-4-ol or tea tree oil, amongst others, as a broader antimicrobial for use on skin, hair, oral cavity, nasal passages, throat and other mucosal surfaces (WO12012385, WO14134709, WO 2015/042410, WO 2022/129773), and treating conditions such as mastitis (WO 2007/094000), halitosis (CN102846791), or as an antimicrobial for protecting and rejuvenating skin (WO 2013/149323).

Additionally, Terpenes, such as carvacrol and Tea tree oil and Terpinen-4-ol, have been used as a biodegradable cleaning product with other ingredients such as probiotics (WO 2022/081174).

A need therefore exists for a method of treating microbial imbalance of the skin and/or mucous membranes with herbal products, which is both more efficacious than prior art herbal compositions and is not associated with any significant adverse effects. Additionally, consideration of targeted anti-microbial function of the herbal products is needed. This includes safer female lower reproductive tract product compositions to aid in women's health are needed, such as products designed to preserve, support, selectively target the vaginal ecosystem homeostasis and healthy function thereof. The present disclosure addresses this need and provide other related advantages.

### BRIEF DESCRIPTION OF THE FIGURES

FIG. 1 are bar graphs depicting the dosage (μl/ml) related selective effect of carvacrol on Gardnerella vaginalis, Candida albicans, Lactobacillus crispus and Lactobacillus gasseri at 24 hours and 48 hours.

FIG. 2 are bar graphs depicting the dosage (μl/ml) related selective effect of Terpinen-4-ol on Gardnerella vaginalis, Candida albicans, Lactobacillus crispus and Lactobacillus gasseri at 24 hours and 48 hours.

FIG. 3 is the result of the checkerboard assays for Lactobacillus crispatus LF6 and Lactobacillus gasseri LF7 at 24 hours and 48 hrs and carvacrol/terpinene-4-ol combinations (synergic and additive) effects for Gardnerella vaginalis LF1 and Candida albicans LF1. Legends: GV—Gardnerella vaginalis, CA—Candida albicans, NP—not performed. Concentrations are in units of μl/ml.

FIG. 4 are bar graphs showing the metabolic activity (%) and biofilm biomass (%) of Gardnerella vaginalis and Candida albicans after treatment with carvacrol, terpinene-4-ol and DMSO.

### DETAILED DESCRIPTION

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or that the alternatives are mutually exclusive, although the disclosure supports a definition that refers to only alternatives and “and/or.” Throughout this application, the term “about” is used to indicate that a value includes the inherent range of variation for the composition, the method being employed to determine the value, or the variation that exists among the study subjects. For example, but not by way of limitation, when the term “about” is utilized, the designated value may vary by plus or minus ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used throughout, ranges are used as a shorthand for describing each and every value that is within the range. All ranges contained within this application are intended to include all numbers, whole or fractions, contained within said range and can be selected as the terminus of the range. When used, the phrase “at least one of” refers to the selection of any one member individually or any combination of the members. The conjunction “and” or “or” can be used in the list of members, but the “at least one of” phrase is the controlling language. For example, at least one of A, B, and C is shorthand for A alone, B alone, C alone, A and B, B and C, A and C, or A and B and C. The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Unless noted otherwise, all percentages in the specification refer to weight percent, where applicable.

As used herein, person, individual, patient, subject, human or the like shall be defined interchangeably, and include animals as defined herein.

As used herein, animals include, but is not limited to mammals which includes but is not limited to rodents, aquatic mammals, domestic animals such as canine and feline, farm animals such as caprine, lagomorph, ovine, porcine, bovine and equine, primate, and various exotic or rare species (e.g., elephant, lion, rhinoceros) and humans. Wherein the terms animal or mammal or their plurals are used, it is contemplated that it also applies to any animals, including, but not limited to, avians, that are capable of the effect exhibited or intended to be exhibited by the context.

As used herein, “effective amount” is preferably an amount that prevents or treats a disease or medical condition in an individual or, more generally, prevents or reduces symptoms, manages progression of the diseases or provides a nutritional, physiological, or medical benefit to the individual. A treatment can be patient-or doctor-related.

As used herein, the terms “treatment,” “treat” and “to alleviate” is preferably to both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the management of a disease or condition or the dietary management for prophylaxis or prevention of a disease or condition.

As used herein, the term “microbial overgrowth” is to be understood to refer to the state in which there is a disruption in the normal balance, a normal, healthy state of co-operation between the microbial population on the skin or mucosal surface, the host cells that form said surface, and immune system cells present on or close to said surface. Such a disruption to the normal balance between these cells (e.g., following pathogen invasion or a change in the local micro-environment, thereby favoring the growth of some microbial species over others), can lead to impaired function of the skin or mucosal surface, as well as initiate an inflammatory response.

As used herein, the term “associated with, or caused by” as used herein is to be understood to refer to the fact that the relevant skin, mucosal or systemic condition is characterized by an absolute and/or relative increase in the population of a certain microbial species. Thus, in some cases, there may be a proven causal relationship between this population increase and the signs and symptoms of the medical condition. In other cases, no such causal relationship can be shown. In all cases referred to herein, however, the change in microbial population on the skin or mucosal surface is co-existent with the observed pathological changes.

As used herein, the term “vaginal microbiota” also referred to as “vaginal flora” or “vaginal ecosystem” refers to the collective microorganisms that normally colonize the vulva and vagina and are non-pathogenic. In general, the vaginal microbiota is predominantly comprised of different strains of Lactobacillus (or related acid-producing bacterial types), which produce lactic acid to keep the vaginal ecosystem as a tightly controlled acidic environment during much of a woman's monthly cycle in reproductive aged women (pH of about 3.5-5.5). Exemplary potential vaginal microbiota species include Lactobacillus acidophilus, Lactobacillus jensenii, Lactobacillus gasseri, Lactobacillus iners, Lactobacillus crispatus, Lactobacillus plantarum, Lactobacillus fermentum, Lactobacillus brevis, Lactobacillus casei, Lactobacillus delbrueckii, Lactobacillus vaginalis, Lactobacillus salivarius. Other lactic-acid producing bacteria that may be part of the vaginal microbiota include species of Atopobium, Leptotrichia, Leuconostoc, Megasphaera, Pediococcus, Streptococcus and Weissella. The species found in normal vaginal microbiota can differ between ethnic groups. Lactobacilli have been shown to inhibit in vitro growth of opportunistic, pathogenic microorganisms, e.g., Bacteroides fragilis, Escherichia coli, Gardnerella vaginalis, Mobiluncus spp., Neisseria gonorrhoeae, Peptostreptococcus anaerobius, Prevotella bivia and Staphylococcus aureus. This inhibitory action is thought to be achieved mainly through the action of lactic acid resulting in modulating pH in niche (Graver and Wade, Ann. Clin. Microbiol. Antimicrob. 70:8, 2011; Matu et al, Anaerobe 7 (5:210, 2010; Skarin and Sylwan, APMIS 94:399, 1986; Strus et al, J. Reprod. Med. 47:41, 2002).

Furthermore, lactobacilli may help prevent opportunistic/pathogenic organisms from adhering to vaginal epithelial cells (Boris and Barbes, Microb. Infect. 2:543, 2000). Other inhibitory mechanisms of lactobacilli include production of hydrogen peroxide and bacteriocins (Martin and Suarez, Appl. Environ. Microbiol. 76:400, 2010; Aroutcheva et al., Am. J. Obstet. Gynecol. 755:375, 2001). While low pH is one factor in lower female reproductive tract homeostasis, the lower female reproductive tract of healthy women is not always maintained at a low pH. The vaginal environment is episodically exposed to natural secretions with a neutral pH (about 7). More specifically, this exposure includes blood flow from the uterus during menses; semen from a partner after sexual intercourse; and fertile cervical mucus production during ovulation. These cyclical disruptions in vaginal pH are transient and rapidly accommodated with a return to an acidic pH level in women with healthy lower female reproductive tract function. Before puberty, during pregnancy and after menopause, a healthy pH of a woman's lower female reproductive tract physiologically rests at a pH of about 4.5 to about 6.8. In short, simple maintenance of an acidic pH does not define lower female reproductive tract homeostasis, but rather a healthy lower female reproductive tract will maintain homeostasis and function while undergoing normal variations in pH depending on a woman's hormone cycle and stage of life, among other factors.

Most healthy women have a stable vaginal microbiota throughout their lives, but a woman's vaginal microbiota can change depending on what reproductive stage of life she is in, e.g., pregnancy, puberty, and menopause. The vaginotropic, “healthy” vaginal microbiota is in large part derived from the woman's mother during her birth. Over 112 different strains of Lactobacillus and related commensal bacteria have been identified in the vagina. Disruptions in the vaginal microbiota and vaginal ecosystem can cause numerous serious diseases, such as bacterial vaginosis, vaginitis, postpartum infections, increased STD rates (van de Wijgert et al., PLoS One 9: el05998, 2014). In short, a stable vaginal microbiota in women contributes to the maintenance of lower female reproductive tract homeostasis, which has significant public health importance.

The present disclosure is therefore primarily directed to a composition and method for treating and/or preventing skin, mucosal, and system conditions resulting from microbial overgrowth, imbalance, or infections, comprising administering a composition to a subject in need of such treatment or prevention, wherein said composition comprises Carvacrol and Terpinen-4-ol at a concentration of Carvacrol in amount of about 0.002% to about 0.008 and Terpinen-4-ol in amount of about 0.25% to about 1.0% and in a ratio of 2:25 to about 8:1000. Further, the synergistic effect of Carvacrol and Terpinen-4-ol to selectively and/or effectively inhibit kinetic growth and biofilm formation or maturation of non-beneficial microorganisms, while one or more of the Commensal Bacteria strains remain unaffected or promoted.

Formulation with ingredients that will aid the actives to adhere on skin or mucosa and do not wash off, so that the actives could be effective in rinse off wash formulations, as well as in leave on applications. Further, formulation with ingredients that will aid the actives to stick on female intimate areas and do not wash off, so that the actives could be effective in rinse off wash formulations, as well as in leave on applications.

The present disclosure is a composition (such as, but not limited to, a topical composition) for balancing commensurable and pathogenic bacteria of the skin (e.g., the skin of the scalp, face, chest, vulvar including labia, and other places in the body) or mucosa (e.g., of the nasopharynx, pharynx, mouth, intimate area, vagina or rectum) utilizing the synergistic effect of Carvacrol and Terpinen-4-ol. The Carvacrol is such that is produced by any supplier, including Alfa Chemistry and Nature Science Technologies. The Terpinen-4-ol is such that is produced by any supplier, including Parchem and Berje. In a non-limiting embodiment of the present disclosure, the composition has Carvacrol in amount of about 0.002% to about 0.008%. In a further embodiment of the present disclosure, the composition has Terpinen-4-ol in amount of about 0.25% to about 1.0%. In an embodiment of the present disclosure, the composition is used on at least one animal. In an embodiment of the present disclosure, the composition is used on at least one mammal. In an embodiment of the present disclosure, the composition is used on at least one human.

In an embodiment of the present disclosure, the composition has Carvacrol and Terpinen-4-ol in a percent ratio of about 4:125 to about 4:200. In a further embodiment of the present disclosure, the composition has Carvacrol and Terpinen-4-ol in a percent ratio of about 1:31.25 to about 1:500. In another embodiment of the present disclosure, the composition has Carvacrol and Terpinen-4-ol in a percent ratio of about 2:625. In an embodiment of the present disclosure, the composition has Carvacrol and Terpinen-4-ol in a percent ratio of about 1:500. In another embodiment of the present disclosure, the composition has Carvacrol and Terpinen-4-ol in a percent ratio of about 1:250. In another embodiment of the present disclosure, the composition has Carvacrol and Terpinen-4-ol in a percent ratio of about 1:125.

In an embodiment of the present disclosure, the composition has about 1% terpinen-4-ol and about 0.008% carvacrol to about 0.5% terpinen-4-ol and about 0.04% carvacrol. In another embodiment of the present disclosure, the composition has about 1% terpinen-4-ol and about 0.008% carvacrol. In a further embodiment of the present disclosure, the composition has about 0.5% terpinen-4-ol and about 0.004% carvacrol. In another embodiment of the present disclosure, the composition has about 0.25% terpinen-4-ol and about 0.002% carvacrol. In a further embodiment of the present disclosure, the composition has about 0.5% terpinen-4-ol and about 0.04% carvacrol.

In an embodiment of the present disclosure, the composition has a pH less than or equal to about 7. In a further embodiment of the present disclosure, the composition has a pH less than 7. In another embodiment of the present disclosure, the composition has a pH about 3.5-about 5.6.

The viscoelastic composition has a low shear viscosity exhibiting a 500,000 cps consistency with the potential to shear degrade to 5 cps under high shear. In an embodiment of the present disclosure, the composition has a viscosity from about 5 centipoise (cps) to about 500,000 cps as measured using a Brookfield™ viscometer with RV5 spindle at 80 RPM at 3 minutes at ambient temperature. In another embodiment of the present disclosure, the composition has a viscosity from about 50 centipoise (cps) to about 250,000 cps. In a further embodiment of the present disclosure, the composition has a viscosity from about 100 centipoise (cps) to about 125,000 cps. In an embodiment of the present disclosure, the composition has a viscosity from about 200 centipoise (cps) to about 80,000 cps. In another embodiment of the present disclosure, the composition has a viscosity from about 300 centipoise (cps) to about 50,000 cps. In an embodiment of the present disclosure, the composition has a viscosity from about 400 centipoise (cps) to about 5,000 cps. In a further embodiment of the present disclosure, the composition has a viscosity of about 1000 cps to about 2500 cps as measured on a Brookfield™ DVII+ viscometer equipped with a temperature regulated disposable sample chamber using a SC4-31 spindle at 180 seconds at 12 RPM at 20° C. In a further embodiment of the present disclosure, the composition has a viscosity from about 490 centipoise (cps) to about 1,100 cps. In another embodiment of the present disclosure, the composition has a viscosity from about 500 centipoise (cps) to about 1,000 cps. In another embodiment of the present disclosure, the composition has a viscosity from about 100 cps to about 500 cps as measured using a Brookfield™ viscometer with LV62 spindle at 50 RPM at ambient temperature.

In an embodiment of the present disclosure, the composition further comprises at least one non-irritating viscosity-increasing agent. In an embodiment of the present disclosure, the composition further comprises at least one non-irritating viscosity-increasing agent wherein the non-irritating viscosity-increasing agent comprises cellulose ether, a carbomer, a polyoxazoline, cetyl hydroxyethylcellulose, carbomer homopolymer type A, carbomer homopolymer type B, carbomer homopolymer type C, hydroxypropyl methyl cellulose (HPMC or hypromellose), or carbomer homopolymer/hypromellose, hydroxyethyl cellulose, hydrophobically-modified hydroxyethyl cellulose, hydroxypropyl cellulose, carboxymethyl cellulose, methyl cellulose, ethylcellulose, ethyl methyl cellulose, hydroxyethyl methyl cellulose, ethyl hydroxyethyl cellulose, guar gum, polyacrylate, acrylate copolymer, acrylate crosspolymer-4, acrylate/Cio-30 alkyl acrylate crosspolymer, carbomer interpolymer Type A, carbomer interpolymer Type B, carbomer copolymer Type A, carbomer copolymer Type B, polycarbophil, polyvinyl alcohol, polyvinylpyrrolidone, polyoxazoline, a combination thereof.

In an embodiment of the present disclosure, the composition does not contain one or more of: thymol, Quercetin, p-cymene, thymoquinone, phenol, eugenol, limonene, aldehyde, parabens, cannabinoids, metal ions, added probiotics, xylose, salvia extract, and combinations thereof.

In an embodiment of the present disclosure, the composition does not contain one or more metal ions such as Zinc ions, copper ions, silver ions, and combinations thereof.

In an embodiment of the present disclosure, the composition does not contain one or more added probiotics such as added lactic acid bacteria probiotics, added Bacillus probiotics, and combinations thereof.

In an embodiment of the present disclosure, the composition does not contain one or more added lactic acid bacteria probiotics such as Carnobacterium sp, Enterococcus sp, Lactobacillus sp, Lactococcus sp, Leuconostoc sp, Oenococcus sp, Streptococcus sp, Tetragenococcus sp, Vagococcus sp, and Weissella sp, and combinations thereof.

In an embodiment of the present disclosure, the composition does not contain one or more added Bacillus probiotics, such as Bacillus licheniformis, Bacillus coagulans, and Bacillus subtilis, and combinations thereof.

In an embodiment of the present disclosure, the composition does not contain thymol. In an embodiment of the present disclosure, the composition does not contain Quercetin. In an embodiment of the present disclosure, the composition does not contain p-cymene. In an embodiment of the present disclosure, the composition does not contain thymoquinone. In an embodiment of the present disclosure, the composition does not contain phenol. In an embodiment of the present disclosure, the composition does not contain eugenol. In an embodiment of the present disclosure, the composition does not contain limonene. In an embodiment of the present disclosure, the composition does not contain aldehyde. In an embodiment of the present disclosure, the composition does not contain parabens. In an embodiment of the present disclosure, the composition does not contain cannabinoids. In an embodiment of the present disclosure, the composition does not contain metal ions. In an embodiment of the present disclosure, the composition does not contain Zinc ions. In an embodiment of the present disclosure, the composition does not contain copper ions. In an embodiment of the present disclosure, the composition does not contain silver ions. In an embodiment of the present disclosure, the composition does not contain added probiotics. In an embodiment of the present disclosure, the composition does not contain added lactic acid bacteria probiotics. In an embodiment of the present disclosure, the composition does not contain added lactic acid bacteria probiotics and wherein the lactic acid bacteria comprises one of more of Carnobacterium sp. Enterococcus sp. Lactobacillus sp. Lactococcus sp. Leuconostoc sp. Oenococcus sp. Streptococcus sp. Tetragenococcus sp. Vagococcus sp. and Weissella sp. In an embodiment of the present disclosure, the composition does not contain added Bacillus probiotics. In an embodiment of the present disclosure, the composition does not contain added Bacillus probiotics and wherein the Bacillus bacteria comprises one of more of Bacillus licheniformis, Bacillus coagulans, and Bacillus subtilis. In an embodiment of the present disclosure, the composition does not contain xylose. In an embodiment of the present disclosure, the composition further contains Quercetin.

The composition may have a strong smell. In an embodiment of the present disclosure the composition further comprises at least one odor controlling or odor neutralizing agent, as is known in the art, to control the odor of the composition.

In an embodiment of the present disclosure, the composition further comprises at least one polymer and its relevant derivatives. The polymer can be at least one of a: biopolymer, sulfonated polymer, sulfonated biopolymer, polymer with mucoadhesive properties, carrageenan, chitosan or combinations thereof. In an embodiment of the present disclosure, the composition further comprises at least one polymer and wherein the polymer is at least one biopolymer. In an embodiment of the present disclosure, the composition further comprises at least one polymer and wherein the polymer is at least one sulfonated polymer. In an embodiment of the present disclosure, the composition further comprises at least one polymer and wherein the polymer is at least one sulfonated biopolymer. In an embodiment of the present disclosure, the composition further comprises at least one polymer and wherein the polymer comprises carrageenan, chitosan, dextran, or combinations thereof. In an embodiment of the present disclosure, the composition further comprises at least one polymer with mucoadhesive properties. In an embodiment of the present disclosure, the composition further comprises at least one polymer and wherein the polymer comprises one or more of: Sulphated polysaccharides composed of uniform or different monomer units of xylose, xylitol, arabinose, rhamnose, fucose, glucose, mannose, galactose, lactose, fructose, glucosamine, galactosamine, mannosamine, glucoronic acid, galacturonic acid, mannuronic acid, carrageenan, agaran, ulvan, fucoids, laminaran, alginate, lentinan, pluran, xylan, dextran, heparin, keratan sulphate, chondroitin-4 and 6-sulfate, dermatan sulfate, heparin sulfate, hyaluronic acid, teichuronic acid, and partial hydrolysates of starch, cellulose, glycogen, chitin, pectin, or combinations thereof. In an embodiment of the present disclosure, the composition further comprises at least one polymer for extended effect of the composition and its derivatives.

In an embodiment of the present disclosure, the composition further comprising at least one surfactant in an amount less than about 40%. In a further embodiment of the present disclosure, the composition further comprising at least one surfactant in an amount about 0.01% to about 20%. In an embodiment of the present disclosure, the composition further comprising at least one surfactant, wherein the surfactant is comprised of one or more of: cetyl hydroxy ethylcellulose, hydrophobically modified hydroxy ethylcellulose, poloxamer, polyoxyethylene glycol alkyl ether, polyoxypropylene glycol alkyl ether, glucoside alkyl ether, polyoxyethylene glycol alkylphenol ether, glycerol alkyl ester, polysorbate, cocamide monoethanolamine (MEA), cocamide diethanolamine (DEA), sodium lauryl ether sulfate, cocoamidopropyl betaine, dodecyldimethylamine oxide, or any combination thereof.

In an embodiment of the present disclosure, the composition further comprising at least one alcohol. In an embodiment of the present disclosure, the composition further comprising at least one alcohol in an amount greater than or equal to about 21.5%. Alternatively, to comply with the US Environmental Protection Agency (EPA) volatile organic compound limits, the composition may comprise at least one alcohol in an amount ranging from about 2% to about 60%.

In an embodiment of the present disclosure, the composition further comprising at least one humectant. In an embodiment of the present disclosure, the composition further comprising at least one humectant and wherein the humectant comprises glycerin, sorbitol, or combinations thereof.

In an embodiment of the present disclosure, the composition further comprising at least one emollient. In an embodiment of the present disclosure, the composition further comprising at least one emollient and wherein the emollient comprises mineral oil, acemannan, cetostearyl alcohol, glyceryl stearate, or combinations thereof.

In an embodiment of the present disclosure, the composition further comprising at least one buffering agent. In an embodiment of the present disclosure, the composition further comprising at least one buffering agent and wherein the buffering agent comprises monocarboxylate or a dicarboxylate. In an embodiment of the present disclosure, the composition further comprising at least one buffering agent and wherein the buffering agent comprises acetate, borate, citrate, fumarate, lactate, malate, malonate, nitrate, phosphate, propanoate, succinate, tartrate, tromethamine, lactic acid, sodium lactate, sodium phosphate, potassium phosphate, sodium citrate, potassium citrate, or any combination thereof.

In an embodiment of the present disclosure, the composition further comprising at least one pH modifying agent. In an embodiment of the present disclosure, the composition further comprising at least one pH modifying agent wherein the pH modifying agent comprises at least one acidifying agent, at least one alkalizing agent, or combinations thereof. In an embodiment of the present disclosure, the composition further comprising at least one pH modifying agent wherein the pH modifying agent is at least one acidifying agent. In an embodiment of the present disclosure, the composition further comprising at least one pH modifying agent wherein the pH modifying agent is at least one acidifying agent wherein the acidifying agent comprises acetic acid, citric acid, fumaric acid, hydrochloric acid, lactic acid, malic acid, nitric acid, phosphoric acid, monobasic potassium phosphate, propionic acid, monobasic sodium phosphate, sulfuric acid, tartaric acid, or combinations thereof. In an embodiment of the present disclosure, the composition further comprising at least one pH modifying agent wherein the pH modifying agent is at least one alkalizing agent wherein the alkalizing agent comprises ammonia, ammonium carbonate, diethanolamine, monoethanolamine, potassium hydroxide, dibasic potassium phosphate, sodium bicarbonate, sodium borate, sodium carbonate, sodium hydroxide, sodium lactate, dibasic sodium phosphate, trolamine, or any combination thereof.

In an embodiment of the present disclosure, the composition further comprises at least one contraceptive agent wherein the composition impairs sperm function, spermicide, thickens cervical mucus, or combinations thereof. In an embodiment of the present disclosure, the composition further comprises at least one contraceptive agent wherein the contraceptive agent impairs sperm function, spermicide, thickens cervical mucus, or combinations thereof.

In an embodiment of the present disclosure, the composition is sterile. In an embodiment of the present disclosure, the composition is preservative-free.

The method of the present disclosure is suitable for use in the case of many type of skin, mucosal or systemic infection, including those associated (causally or otherwise) with fungi, bacteria and/or viruses. In an embodiment of the present disclosure, the composition is for maintaining bacterial population by targeted control of the skin, mucosa, or combinations thereof. In an embodiment of the present disclosure, the composition is for bacterial population balance of the skin by targeted control. In an embodiment of the present disclosure, the composition is for bacterial population balance of mucosa by targeted control. In an embodiment of the present disclosure, the composition is for bacterial balance is not of the gastrointestinal mucosa. In an embodiment of the present disclosure, the composition is for bacterial balance is not of the pulmonary mucosa.

In another embodiment, the method is used to treat or prevent a skin, mucosal or system infection associated with, or caused by, a bacteria. In some embodiments, the bacteria is Staphylococcus aureus. In some particular embodiments, the bacterial organism is a methicillin-resistant Staphylococcus aureus.

In an embodiment of the present disclosure, the composition is for maintaining or restoring bacterial population balance of intimate areas, such as the female intimate areas. In a further embodiment of the present disclosure, the composition is for bacterial population balance of the female intimate such as one or more of: vagina, vulva, including labia, external cervix, perineum, external genitalia, and combinations thereof. In an embodiment of the present disclosure, the composition is for bacterial balance of intimate areas. In an embodiment of the present disclosure, the composition is for bacterial balance of female intimate areas. In an embodiment of the present disclosure, the composition is for bacterial balance of vagina. In an embodiment of the present disclosure, the composition is for bacterial balance of vulva. In an embodiment of the present disclosure, the composition is for bacterial balance of the perineum. In an embodiment of the present disclosure, the composition is for bacterial balance of the external genitalia. In an embodiment of the present disclosure, the composition is for bacterial balance of external cervix.

In an embodiment of the present disclosure, the composition prevents infection. In an embodiment of the present disclosure, the composition prevents bacterial infection. In an embodiment of the present disclosure, the composition prevents bacterial vaginosis infection. In an embodiment of the present disclosure, the composition prevents bacterial vaginosis infection such as Gardenella vaginalis infection, Mobiluncus infection, Prevotella infection, Mycoplasma hominis infection, Atopobium vaginae infection, and combinations thereof. In an embodiment of the present disclosure, the composition prevents Gardenella vaginalis infection. In an embodiment of the present disclosure, the composition prevents Mobiluncus infection. In an embodiment of the present disclosure, the composition prevents Prevotella infection. In an embodiment of the present disclosure, the composition prevents Mycoplasma hominis infection. In an embodiment of the present disclosure, the composition prevents Atopobium vaginae infection.

In an embodiment of the present disclosure, the composition is for bacterial population balance of one or more of: nasopharynx, nasal mucosa, pharyngeal mucosa, or combinations thereof. In an embodiment of the present disclosure, the composition is for bacterial balance of nasopharynx. In an embodiment of the present disclosure, the composition is for bacterial balance of nasal mucosa. In an embodiment of the present disclosure, the composition is for bacterial balance of the pharyngeal mucosa.

In an embodiment of the present disclosure, the composition prevents infection of the nasal pharynx. In an embodiment of the present disclosure, the composition prevents infection of the nasal pharynx like Fungal sinusitis, eosinophilic fungal rhinosinusitis (EFRS), eosinophilic mucinous rhinosinusitis (EMRS), caused by at least one of thrush, Streptococcus sp, Staphylococcus aureus, Mycoplasma pneumoniae, influenza, rhinovirus, Aspergillus, Mucormycosis, Metarrhizium anisopliae, Malassezia or combinations thereof.

In an embodiment of the present disclosure, the composition prevents infection of the pharynx. In an embodiment of the present disclosure, the composition prevents infection of the pharynx caused by at least one of: thrush, Streptococcus sp, Staphylococcus aureus, Mycoplasma pneumoniae, influenza, rhinovirus, Aspergillus, Mucormycosis, Metarrhizium anisopliae, Malassezia or combinations thereof.

In some embodiments, the fungal organism is a yeast. The method of the present disclosure may be used to treat or prevent skin infections caused by, or associated with, many different yeast organisms. In some cases, the dominant yeast associated with the disease is of the Malassezia genus. In many such cases, the Malassezia yeast is a commonly occurring species such as M. furfur, M. globosa, M. slooffiae, M. restricta, and others. A very commonly found yeast in skin conditions such as seborrheic dermatitis is M. furfur. In other cases, the method of the present disclosure is used to treat and/or prevent overgrowth, infections and diseases associated with the opportunist pathogenic yeast Candida albicans. In some embodiments, the method of the present disclosure may be used to treat and/or prevent overgrowth, infections and diseases associated with fluconazole-resistant strains of C. albicans.

In an embodiment of the present disclosure, the composition prevents fungal infection. In an embodiment of the present disclosure, the composition prevents fungal infection such as at least one of: Candidiasis, Cryptococcosis, Aspergillosis, Coccidioidomycosis, Histoplasmosis, Blastomycosis, and combinations thereof. In an embodiment of the present disclosure, the composition prevents Candidiasis from at least one of: Candida albicans, C. glabrata, C. parapsilosis, C. tropicalis, Candida auris and combinations thereof. In an embodiment of the present disclosure, the composition prevents Cryptococcosis from at least one of: Cryptococcus neoformans, C. gattii, and combinations thereof. In an embodiment of the present disclosure, the composition prevents Aspergillosis. In an embodiment of the present disclosure, the composition prevents Coccidioidomycosis from at least one of: Coccidioides immitis, C. posadasii, and combinations thereof. In an embodiment of the present disclosure, the composition prevents Histoplasmosis from at least one of: Histoplasma capsulatum, Histoplasma gondii, and combinations thereof. In an embodiment of the present disclosure, the composition prevents Blastomycosis. In an embodiment of the present disclosure, the composition prevents fungal vaginosis infection. In an embodiment of the present disclosure, the composition prevents fungal vaginosis infection from Candida albicans.

In another embodiment, the present disclosure utilizes botanical anti-microbial that would effectively remove pathogenic micro-organisms without affecting the “friendly” microbiome in our body, which are beneficial species to boost our immune system and health. In an embodiment of the present disclosure, the composition promotes, maintains, but does not disrupt, healthy growth of one or more of: Lactobacillus gasseri, Lactobacillus crispatus, Lactobacillus acidophilus, Lactobacillus jensenii, Lactobacillus iners, Lactobacillus plantarum, Lactobacillus fermentum, Lactobacillus brevis, Lactobacillus casei, Lactobacillus delbrueckii, Lactobacillus vaginalis, Lactobacillus salivarius, Atopobium, Leptotrichia, Leuconostoc, Megasphaera, Pediococcus, and combinations thereof. In an embodiment of the present disclosure, the composition promotes healthy growth of Lactobacillus gasseri. In an embodiment of the present disclosure, the composition promotes healthy growth of Lactobacillus crispatus. In an embodiment of the present disclosure, the composition promotes healthy growth of Lactobacillus acidophilus. In an embodiment of the present disclosure, the composition promotes healthy growth of Lactobacillus jensenii. In an embodiment of the present disclosure, the composition promotes healthy growth of Lactobacillus iners. In an embodiment of the present disclosure, the composition promotes healthy growth of Lactobacillus plantarum. In an embodiment of the present disclosure, the composition promotes healthy growth of Lactobacillus fermentum. In an embodiment of the present disclosure, the composition promotes healthy growth of Lactobacillus brevis. In an embodiment of the present disclosure, the composition promotes healthy growth of Lactobacillus casei. In an embodiment of the present disclosure, the composition promotes healthy growth of Lactobacillus delbrueckii. In an embodiment of the present disclosure, the composition promotes healthy growth of Lactobacillus vaginalis. In an embodiment of the present disclosure, the composition promotes healthy growth of Lactobacillus salivarius. In an embodiment of the present disclosure, the composition promotes healthy growth of Atopobium. In an embodiment of the present disclosure, the composition promotes healthy growth of Leptotrichia. In an embodiment of the present disclosure, the composition promotes healthy growth of Leuconostoc. In an embodiment of the present disclosure, the composition promotes healthy growth of Megasphaera. In an embodiment of the present disclosure, the composition promotes healthy growth of Pediococcus.

In one embodiment of the method of the present disclosure, the composition is applied topically to the skin or mucosa. In an embodiment of the present disclosure, the composition is applied or ingested in one or more of: a topical application, aerosol, caplet, capsule, cream, drop, emulsion, film, foam foaming wash, gel, jelly, liquid, lotion, lube (e.g. personal, sexual, anal, vaginal hydration, and the like), mucilage, oil, ointment, paste, powder, semi-solid, serum, shampoo, spray, suppository, suspension, tablet, wash, and the like, and combinations thereof. In an embodiment of the present disclosure, the composition is applied in a topical application. In an embodiment of the present disclosure, the composition is applied in a caplet. In an embodiment of the present disclosure, the composition is applied in a capsule. In an embodiment of the present disclosure, the composition is applied in a cream. In an embodiment of the present disclosure, the composition is applied in a drop. In an embodiment of the present disclosure, the composition is applied in a film. In an embodiment of the present disclosure, the composition is applied in a foam. In an embodiment of the present disclosure, the composition is applied in a foaming wash. In an embodiment of the present disclosure, the composition is applied in a gel. In an embodiment of the present disclosure, the composition is applied in a jelly. In an embodiment of the present disclosure, the composition is applied in a liquid. In an embodiment of the present disclosure, the composition is applied in a lube. In an embodiment of the present disclosure, the composition is applied in an oil. In an embodiment of the present disclosure, the composition is applied in a ointment. In an embodiment of the present disclosure, the composition is applied in a paste. In an embodiment of the present disclosure, the composition is applied in a powder. In an embodiment of the present disclosure, the composition is applied in a semi-solid. In an embodiment of the present disclosure, the composition is applied in a shampoo. In an embodiment of the present disclosure, the composition is applied in a spray. In an embodiment of the present disclosure, the composition is applied in a suppository. In an embodiment of the present disclosure, the composition is applied in a tablet. In an embodiment of the present disclosure, the composition is applied in a wash. In this embodiment, the composition may be formulated in a form that includes, but is not limited to, as an aerosol, caplets, capsules, cream, drops, emulsion, film, foam, foaming wash, gel, jelly, liquid, lotion, lube (personal, sexual, anal, vaginal hydration, and the like), mucilage, foam, oil, ointment, paste, powder, semi-solid, serum, shampoo, spray, suppository, suspension, tablets, and/or wash, and is applied topically to the skin or mucosa (e.g., the mucosa of the nasal cavity, oral cavity, vulva, or rectum).

In some embodiments, the composition of the present disclosure is used to treat or prevent skin, mucosal and systemic conditions, directly or indirectly resulting from microbial overgrowth, imbalance or infections which are related to, or caused by, a microbial agent selected from the group consisting of fungi, bacteria and viruses. The present disclosure includes a method for treating and/or preventing skin, mucosal and systemic conditions resulting from microbial overgrowth, imbalance or infections comprising administering the compositions disclosed herein and obvious variants of these compositions. The method of the present disclosures contemplates that microbial imbalance is caused by one or more of: at least one bacteria, at least one fungus, at least one virus, or combinations thereof. The method of the present disclosures includes applying the compositions topically to the skin, mucosa, or both.

Alterations in the microecological balance of the reproductive tract can result in dysbiosis, inappropriate inflammatory responses, and abnormal immune responses, all of which can contribute to a wide range of health problems. The present disclosure includes a method for treating or preventing an infection of the lower reproductive tract, by administering the composition to the lower reproductive tract of a female subject. The present disclosure includes a method for enhancing vaginal ecosystem homeostasis of the lower reproductive tract, by administering the composition to the lower reproductive tract of a female subject.

In an embodiment of the present disclosure, the composition controls odor. In an embodiment of the present disclosure, the composition prevents discharge. In an embodiment of the present disclosure, the composition prevents pain, discomfort, or pain and discomfort.

In an embodiment of the present disclosure, the composition prevents or makes the user less susceptible to viral infection. In an embodiment of the present disclosure, the composition prevents or makes the user less susceptible to Human herpes simplex virus infection. In an embodiment of the present disclosure, the composition prevents or makes the user less susceptible to Human papilloma virus infection. In an embodiment of the present disclosure, the composition prevents or makes the user less susceptible to sexually transmitted infections. In an embodiment of the present disclosure, the composition prevents or makes the user less susceptible to Chlamydia trachomatis. In an embodiment of the present disclosure, the composition prevents or makes the user less susceptible to Neisseria gonorrhoeae.

In an embodiment of the present disclosure, the composition prevents biofilm formation or maturation. In an embodiment of the present disclosure, the composition prevents biofilm formation or maturation from at least one of Gardenella vaginalis, Candida albicans, or combinations thereof. In an embodiment of the present disclosure, the composition prevents biofilm formation or maturation from Gardenella vaginalis. In an embodiment of the present disclosure, the composition prevents biofilm formation or maturation from Candida albicans.

In an embodiment of the present disclosure, the composition is non-irritating. In an embodiment of the present disclosure, the composition does not cause discomfort. In an embodiment of the present disclosure, the composition enhances fertility. In an embodiment of the present disclosure, the composition decreases fertility.

In an embodiment of the present disclosure, the composition helps to restore or maintain a healthy bacterial population balance where there is potential for disruption of a healthy bacterial population balance. In an embodiment of the present disclosure, the composition helps to restore or maintain a healthy bacterial balance where there is potential for disruption a healthy bacterial balance caused by one or more of: side effect from medication, antibiotic, antimicrobial, a side effect from medical treatment, hormone imbalance, coitus, sexual activity, and combinations thereof. In an embodiment of the present disclosure, the composition helps to restore or maintain a healthy bacterial balance where there is potential for disruption caused by a side effect from medication such as oral contraceptives, antidepressants. In an embodiment of the present disclosure, the composition helps to restore or maintain a healthy bacterial balance where there is potential for disruption caused by an antibiotic. In an embodiment of the present disclosure, the composition helps to restore or maintain a healthy bacterial balance where there is potential for disruption caused by a side effect from medical treatment such as radiation. In an embodiment of the present disclosure, the composition helps to restore or maintain a healthy bacterial balance where there is potential for disruption caused by medical conditions, such as Endometriosis, autoimmune disease (i.e. Rheumatic disease), diabetes, Obesity, and combinations thereof. In an embodiment of the present disclosure, the composition helps to restore or maintain a healthy bacterial balance where there is potential for disruption caused by lifestyle, such as exercise, feminine hygiene practices (douching), diet, sexual activity, smoking, stress, alcohol consumption, steam rooms, saunas, climate, perimenopause, menopause, climacteric, pregnancy.

In an embodiment of the present disclosure, the composition helps to maintain a healthy bacterial balance during one or more of: ovulation, menstruation, perimenopause, menopause, post-menopause, or combinations thereof. In an embodiment of the present disclosure, the composition helps to maintain a healthy bacterial balance during ovulation. In an embodiment of the present disclosure, the composition helps to maintain a healthy bacterial balance during menstruation. In an embodiment of the present disclosure, the composition helps to maintain a healthy bacterial balance in perimenopause. In an embodiment of the present disclosure, the composition helps to maintain a healthy bacterial balance in menopause. In an embodiment of the present disclosure, the composition helps to maintain a healthy bacterial balance in post-menopause.

In a still further aspect, the present disclosure is directed to the use of a composition as defined hereinabove in the manufacture of a medicament for treating and/or preventing microbial infections of the skin or mucosa or for treating and/or preventing systemic infections. All of the technical features described above in relation to other aspects of the present disclosure also apply equally to this aspect.

De-ionized water (59.912 w/w %), 2EO Sodium Lauryl Ether Sulphate (12.16 w/w %), cocoamidopropyl betaine (4.45% w/w), Akypto soft 100 BVC (4.85% w/w), 98% sodium chloride (2% w/w), 99.7% USP Glycerine (10% w/w), 50% L-Lactic acid (4.5% w/w), sodium hydroxide (0.624% w/w), dissolvine GL-47-S (0.4% w/w), Microcare SBB (0.6% w/w), xanthum gum (0.1% w/w), 22erpinene-4-ol (0.25%-1% w/w), carvacrol (0.002%-0.008% w/w).

Embodiments and/or features therein may be freely combined with one another.

### EXAMPLES

The present disclosure is further described in the following examples. The examples are merely illustrative and do not in any way limit the scope of the present disclosure as described and claimed.

| Raw material | Range (%) | & W/W | % W/W | % W/W | % W/W | % W/W | % W/W | % W/W | % W/W |
|---|---|---|---|---|---|---|---|---|---|
| Carvecrol | 0.004~0.008 | 0.004 | 0.004 | 0.008 | 0.008 | 0.004 | 0.004 | 0.008 | 0.008 |
| Terpinol-1 | 0.5~1 | 0.5 | 1 | 0.5 | 1 | 0.5 | 1 | 0.5 | 1 |
| SOD. LAURYL ETHER SULFATE, 2EO | 1~25 | 12.16 | 12.16 | 12.16 | 12.16 | 12.16 | 12.16 | 12.16 | 12.16 |
| COCOAMIDOPROPYL BETAINE | 0-5 | 4.45 | 4.45 | 4.45 | 4.45 | 4.45 | 4.45 | 4.45 | 4.45 |
| AKYPO SOFT 100 BVC | 0-5 | 4.85 | 4.85 | 4.85 | 4.85 | 4.85 | 4.85 | 4.85 | 4.85 |
| AMIDET N | 0-5 | 1.7 | 1.7 | 1.7 | 1.7 | 1.7 | 1.7 | 1.7 | 1.7 |
| SODIUM CHLORIDE | 0-5 | 2 | 2 | 2 | 2 | 2 | 2 | 2 | 2 |
| GLYCERINE USP | 0-5 | 10 | 10 | 10 | 10 | 10 | 10 | 10 | 10 |
| L-LACTIC ACID | 0.2~10 | 4.5 | 4.5 | 4.5 | 4.5 | 4.5 | 4.5 | 4.5 | 4.5 |
| SODIUM HYDROXIDE | 0-5 | 0.624 | 0.624 | 0.624 | 0.624 | 0.624 | 0.624 | 0.624 | 0.624 |
| DISSOLVINE GL-47-5/EDTA | 0-5 | 0.4 | 0.4 | 0.4 | 0.4 | 0.4 | 0.4 | 0.4 | 0.4 |
| Sodium Benzoate (benzoic acid) | 0.2~5 | 0.6 | 0.6 | 0.6 | 0.6 | 0.6 | 0.6 | 0.6 | 0.6 |
| Coconut oil | 0-5 | | | | | | | | |
| Lactobionic acid | 0-5 | | | | | | | | |
| chamomile | 0-5 | | | | | | | | |
| vitamin B12 | 0-5 | | | | | | | | |
| vitamin D | 0-5 | | | | | | | | |
| ectoine | 0-5 | | | | | | | | |
| Quercertin | 0.002~0.016 (20 μg/ml ~160 | | | | | 0.002 | 0.002 | 0.002 | 0.002 |
| green tea | 0-5 | | | | | | | | |
| aloe vera | 0-5 | | | | | | | | |
| cucumber extracts | 0-5 | | | | | | | | |
| avocado oil | 0-5 | | | | | | | | |
| chamomile | 0-5 | | | | | | | | |
| white tea | 0-5 | | | | | | | | |
| Hyaluronic acid | 0-5 | | | | | | | | |
| prickly pear oil | 0-5 | | | | | | | | |
| new Fragrance | 0-5 | | | | | | | | |
| DEIONIZED WATER | 0-70 | 58.212 | 57.712 | 56.208 | 57.708 | 58.21 | 57.71 | 58.206 | 57.706 |
| Total Vol. | 100 | 100 | 100 | 100 | 100 | 100 | 100 | 100 | 100 |

| Raw material | % W/W | % W/W | % W/W | % W/W | % W/W | % W/W | % W/W | % W/W | % W/W | % W/W |
|---|---|---|---|---|---|---|---|---|---|---|
| Carvecrol | 0.004 | 0.004 | 0.008 | 0.008 | 0.004 | 0.004 | 0.008 | 0.008 | 0.004 | 0.004 |
| Tarpinol-1 | 0.5 | 1 | 0.5 | 1 | 0.5 | 1 | 0.5 | 1 | 0.5 | 1 |
| SOD. LAURYL ETHER SULFATE, 2EO | 12.16 | 12.16 | 12.16 | 12.16 | 12.16 | 12.16 | 12.16 | 12.16 | 12.16 | 12.16 |
| COCOAMIDOPROPYL BETAINE | 4.45 | 4.45 | 4.45 | 4.45 | 4.45 | 4.45 | 4.45 | 4.45 | 4.45 | 4.45 |
| AKYPO SOFT 100 BVC | 4.85 | 4.85 | 4.85 | 4.85 | 4.85 | 4.85 | 4.85 | 4.85 | 4.85 | 4.85 |
| AMIDET N | 1.7 | 1.7 | 1.7 | 1.7 | 1.7 | 1.7 | 1.7 | 1.7 | 1.7 | 1.7 |
| SODIUM CHLORIDE | 2 | 2 | 2 | 2 | 2 | 2 | 2 | 2 | 2 | 2 |
| GLYCERINE USP | 10 | 10 | 10 | 10 | 10 | 10 | 10 | 10 | 10 | 10 |
| L-LACTIC ACID | 4.5 | 4.5 | 4.5 | 4.5 | | 4.5 | | | | 4.5 |
| SODIUM HYDROXIDE | 0.624 | 0.624 | 0.624 | 0.624 | 0.624 | 0.624 | 0.624 | 0.624 | 0.624 | 0.624 |
| DISSOLVINE GL-47-S/EDTA | 0.4 | 0.4 | 0.4 | 0.4 | 0.4 | 0.4 | 0.4 | 0.4 | 0.4 | 0.4 |
| Sodium Benzoate (benzoic acid) | 0.6 | 0.6 | 0.6 | 0.6 | 0.6 | 0.6 | 0.6 | 0.6 | 0.6 | 0.6 |
| Coconut oil | | | | | 0.1 | | | | 0.1 | |
| Lactobionic acid | | | | | 0.1 | | | | 0.1 | |
| chamomile | | | | | 0.1 | | | | 0.1 | |
| vitamin B12 | | | | | 0.1 | | | | 0.1 | |
| vitamin D | | | | | | | | | | |
| ectoine | | | | | | | | | | |
| Quercertin | 0.016 | 0.016 | 0.016 | 0.016 | | | | | 0.002 | 0.015 |
| green tea | | | | | | 0.1 | | | | 0.1 |
| aloe vera | | | | | | | | | | |
| cucumber extracts | | | | | | 0.1 | | 0.1 | | 0.1 |
| avocado oil | | | | | | | | | | |
| chamomile | | | | | | | | | | |
| white tee | | | | | | | | | | |
| Hyaluronic acid | | | | | | | 0.1 | | | |
| prickly pear oil | | | | | | | 0.1 | | | |
| new Fragrance | | | | | | | | 0.2 | | |
| DEIONIZED WATER | 58.196 | 57.596 | 58.192 | 57.692 | 62.312 | 57.512 | 62.508 | 61.908 | 62.31 | 57.495 |
| Total Vol. | 100 | 100 | 100 | 100 | 100 | 100 | 100 | 100 | 100 | 100 |

| Raw material | % W/W | % W/W | % W/W |
|---|---|---|---|
| Carvecrol | 0.008 | 0.008 | 0.008 |
| Terpinol-1 | 0.5 | 1 | 1 |
| SOD. LAURYL ETHER SULFATE, 2EO | 12.16 | 12.16 | 12.16 |
| COCOAMIDOPROPYL BETAINE | 4.45 | 4.45 | 4.45 |
| AKYPO SOFT 100 BVC | 4.85 | 4.85 | 4.85 |
| AMIDET N | 1.7 | 1.7 | 1.7 |
| SODIUM CHLORIDE | 2 | 2 | 2 |
| GLYCERINE USP | 10 | 10 | 10 |
| L-LACTIC ACID | | | |
| SODIUM HYDROXIDE | 0.624 | 0.624 | 0.624 |
| DISSOLVINE GL-47-S/EDTA | 0.4 | 0.4 | 0.4 |
| Sodium Benzoate (benzoic acid) | 0.6 | 0.6 | 0.6 |
| Coconut oil | | | 0.1 |
| Lactobionic acid | | | 0.1 |
| chamomile | | | 0.1 |
| vitamin B12 | | | 0.1 |
| vitamin D | | | 0.1 |
| ectoine | | | 0.1 |
| Quercertin | 0.002 | 0.016 | 0.016 |
| green tea | | | 0.1 |
| aloe vera | | | 0.1 |
| cucumber extracts | | 0.1 | 0.1 |
| avocado oil | | | 0.1 |
| chamomile | | | 0.1 |
| white tea | | | 0.1 |
| Hyaluronic acid | 0.1 | | 0.1 |
| prickly pear oil | 0.1 | | 0.1 |
| new Fragrance | | 0.2 | 0.2 |
| DEIONIZED WATER | 62.506 | 61.892 | 60.592 |
| Total Vol. | 100 | 100 | 100 |

The aim was to utilize the selective antimicrobial efficacy of essential oil ingredients, so that pathogenic microbes are inhibited whereas healthy microbiome maintenance, and balance is possible. Additionally, natural plant-based actives would resolve the fight against microbial resistance to the current microbicidals (e.g., antibiotics), and would be easily accepted by the consumers.

The strains used were Lactobacillus crispatus LF6 and Lactobacillus gasseri LF7), one strain of Gardnerella vaginalis LF1 and one strain of Candida albicans LF, that correspond to clinical isolates collected from women vaginal cavity. The antimicrobial activity method was based on the current standards described by EUCAST (European Committee on Antimicrobial Susceptibility Testing) and ISO 20776-1 (2006) and ISO 20776-2-(2007) for determining the minimum inhibitory concentration (MIC). Terpinen-4-ol and Carvacrol were emulsified with DMSO (up to 1% (v/v)) in culture medium to obtain a concentration corresponding to 2× higher than the testing one. Sequential dilutions (1:2) were prepared in a sterile 96 wells plate with culture medium. All preparations were performed in aseptic conditions. For each of the ingredients tested, 8 concentrations were tested. The concentration range tested were 2.50-0.02 μL/mL for carvacrol and 80-0.63 μL/mL (for Lactobacillus spp. and C. albicans) and 5-0.04 μL/mL (G. vaginalis) for terpinen-4-ol.

In parallel, calibrated suspensions of microorganisms were prepared with a cell density of 1.0 on the McFarland scale (Lactobacillus spp. and Candida albicans) and 0.13 absorbance (Gardnerella vaginalis), corresponding to 1×10⁸ CFU/mL for bacteria (Lactobacillus spp. and Gardnerella vaginalis) and 1×10⁶ CFU/mL for yeast (Candida albicans). De Man, Rogosa and Sharpe Broth (MRSB) media was used for growth of Lactobacillus spp., New York City III Broth (NYCIIIB) media and Roswell Park Memorial Institute 1640 (RPMI-1640) media was used respectively to grow Gardnerella vaginalis and Candida albicans. After adding the microorganism suspensions to the compound dilutions in a 1:1 ratio (ingredient dilution/microorganism suspension), the number of microorganisms to be tested were approximately 5×10⁵ CFU/mL for bacteria (Lactobacillus spp. and Gardnerella vaginalis) and 5×10⁴ CFU/mL for yeast (Candida albicans).

After homogenization of the working microbial suspension with dilutions of the two ingredients, the 96-well plates were incubated according to the incubation conditions suitable for each microorganism. For, L. crispatus and L. gasseri, incubation was done at 37° C. for 24 and 48h in microaerophilic atmosphere. For G. vaginalis, incubation was done at 37° C. for 24 and 48h in microaerophilic atmosphere (10% CO2, 5% O2 and 85% N2), and for C. albicans, incubation was done at 32.5° C., for 24 and 48 h in aerophilic atmosphere (>20% O2). After incubation, the wells were evaluated macroscopically to check for microorganism growth. Turbid wells indicated the presence of microbial growth, and clear wells indicated the absence of microbial growth. The lowest concentration tested in which wells without turbidity, correspondent to the minimum inhibitory concentration (MIC).

To validate the test, three negative controls (sterility control of the culture medium; study substance dilutions with culture medium; emulsifier with culture medium) and two positive controls (growth control for each microorganism using culture media and growth control for each microorganism using emulsifier with culture media) were performed. Additional control was used to demonstrate that the concentration of DMSO (emulsifier) in the media does not affect cell viability.

The antimicrobial activity in a double-dose response (checkerboard) was determined for Carvacrol and Terpinen-4-ol on Lactobacillus crispatus LF6, Lactobacillus gasseri LF7, Gardnerella vaginalis LF1 and Candida albicans LF1. A serial dilution of Carvacrol and Terpinen-4-ol were prepared individually and then combined. Next, the combination plates were placed in contact with calibrated suspensions of the microorganisms. After homogenization, the microorganisms were incubated under favorable growth conditions. The bacteria were incubated in a microaerophilic atmosphere (10% CO2, 5% O2, and 85% N2) at 37° C.±1° C. for 48 hours, and Candida albicans in an aerophilic atmosphere (>20% O2) at 32.5° C.±2.5° C. for 48 hours. The lowest concentration of the test substance combination at which no visible growth occurs, that is minimum inhibitory concentration (MIC) was determined. Two independent assays will be performed, presenting concordant results.

To determine the effect of a tested combination, the fractional inhibitory concentration index (FIC index) was calculated according to the following formula:

FIC index=FIC A+FIC B=(MIC combination A/MIC A)+(MIC combination B/MIC B)
where:
MIC A/MIC B—MIC value obtain when study substance A/B are tested alone;
MIC combination A—MIC value of study substance A in the presence of study substance B;
MIC combination B—MIC value of study substance B in the presence of study substance A.

The interaction between the study substances can be classified as synergistic, indifferent, or antagonistic, based on the FIC index value. If:
FIC index≤0.5—interaction is synergistic;
FIC index>0.5 and ≤1.0—interaction is additive;
FIC index>1.0 and ≤4.0—interaction is indifferent;
FIC index>4.0—interaction is antagonistic.

For, the biofilm prevention assay, the effect of carvacrol and terpinen-4-ol on the biofilm formation of Gardnerella vaginalis and Candida albicans were checked. A pre-inoculum was made by growing the strains in broth culture media, Gardnerella vaginalis in NYCIIIB and Candida albicans in Yeast Peptone Dextrose Broth (YPDB). The Gardnerella vaginalis was incubated in a microaerophilic atmosphere (10% CO2, 5% O2, and 85% N2) at 37° C.±1° C. for 48 hours and Candida albicans in an aerophilic atmosphere (>20% O2) at 32.5° C.±2.5° C. for 48 hours. The cellular density was adjusted to 1×10⁶ CFU/mL for Candida albicans and to 1×10⁸ CFU/mL for Gardnerella vaginalis. The two ingredients were directly mixed with culture media and DMSO to 2× the desired concentration. Sequential dilutions (1:2) were prepared in a sterile 96-well plate with culture media. All preparations were performed in aseptic conditions. DMSO (upto 1% (v/v)) was used for the mixture preparation, and an additional control was performed to demonstrate that this concentration does affect cell viability. Serial dilutions of each of terpinen-4-ol and carvacrol were placed in contact with a calibrated suspension (1×10⁸ bacteria/ml or 1×10⁶ yeast/mL) of the microorganisms. After incubation under favorable growth conditions for each microorganism as above, the biofilm metabolic activity and biomass were determined. The results are expressed in percentages of viability based on the control without the study substances.

To determine biofilm biomass, the biofilms were fixed with methanol for 30 minutes and stained with crystal violet (0.02%, w/v) for 30 minutes at room temperature. The excess of crystal violet was removed with distilled water, and bound crystal violet was released by adding 33% (v/v) acetic acid solution. The absorbance was read at 600 nm.

Biofilm metabolic activity was determined by incubating the formed biofilms with culture medium supplemented with 1 mg/mL MTT for 3 hours. Absorbance was measured at 560 nm after resuspending the formazan crystals with DMSO.

The negative control was normalized at 100% biomass and cell viability, and the absorbance values obtained in each assay were normalized considering the absorbance values of the negative control. The results were expressed as percentages of viability.

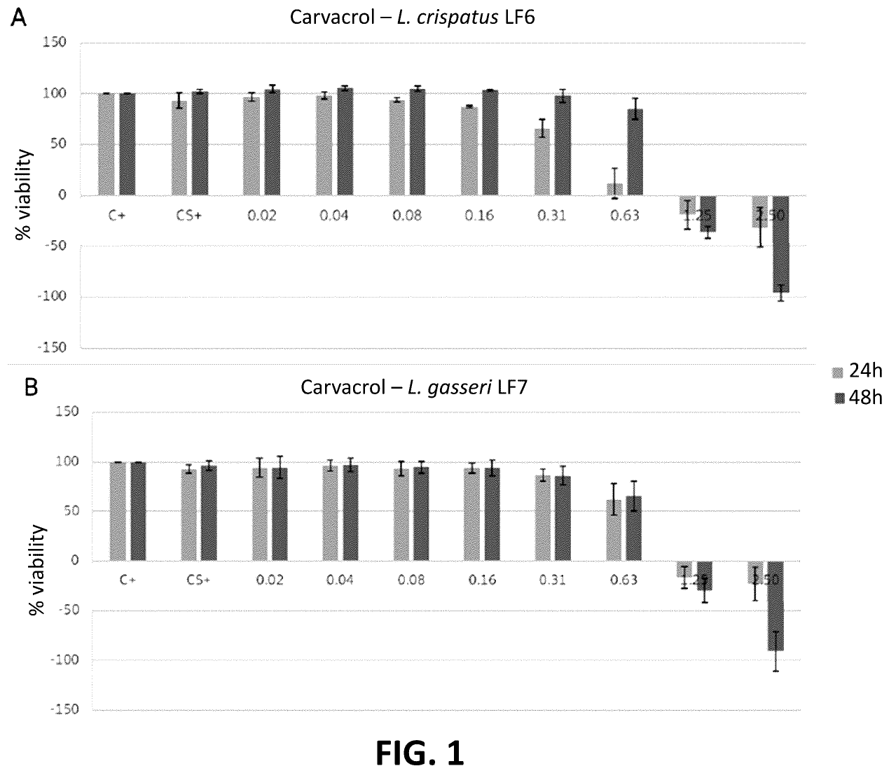

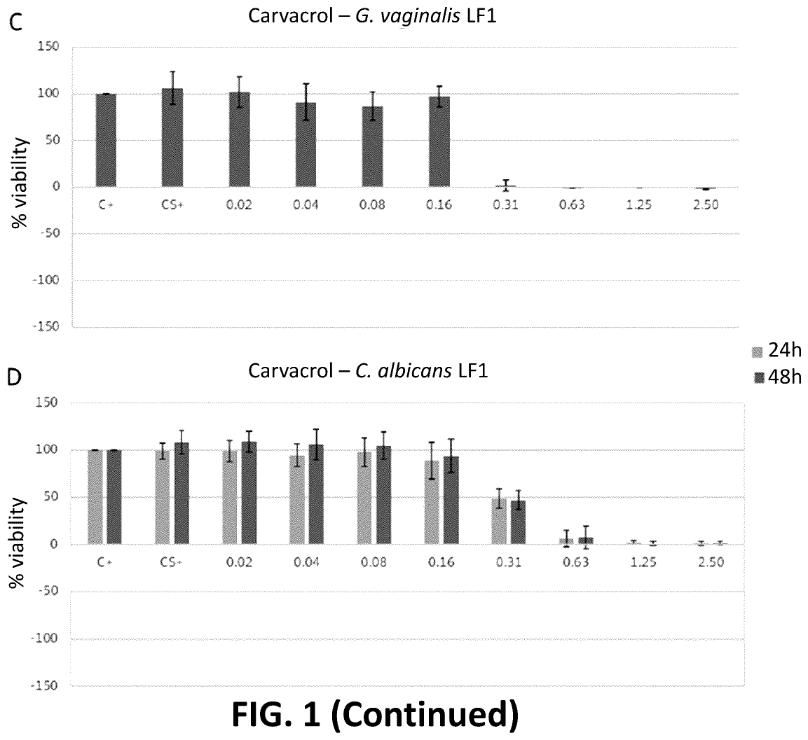

FIG. 1 shows that the MIC of Carvacrol for Gardenella vaginalis LF1 was 0.31 μl/ml, which also showed 50% inhibition for C. albicans LF1, while the Lactobacillus strains remained unaffected at that dose.

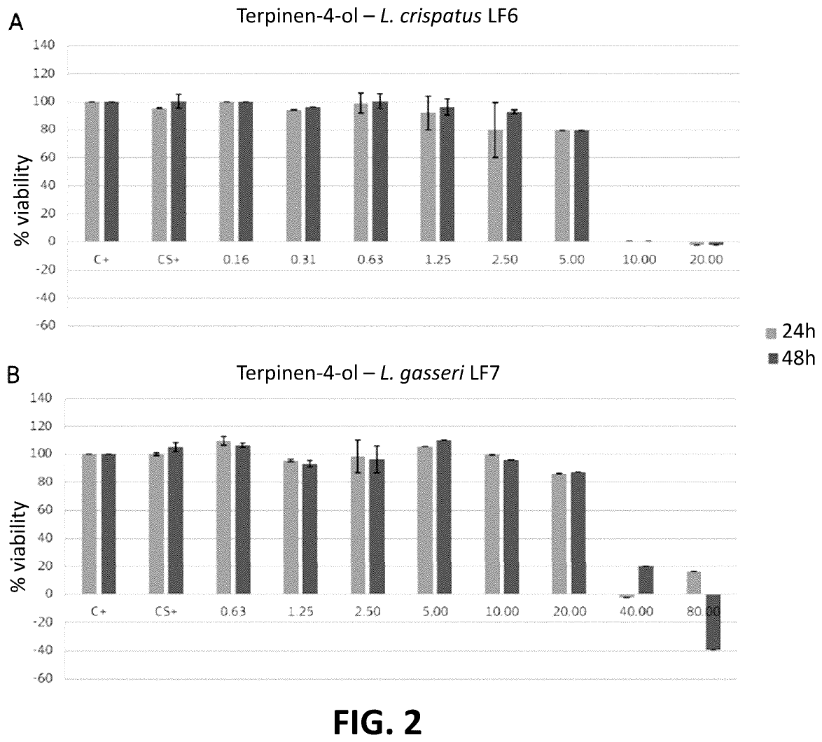

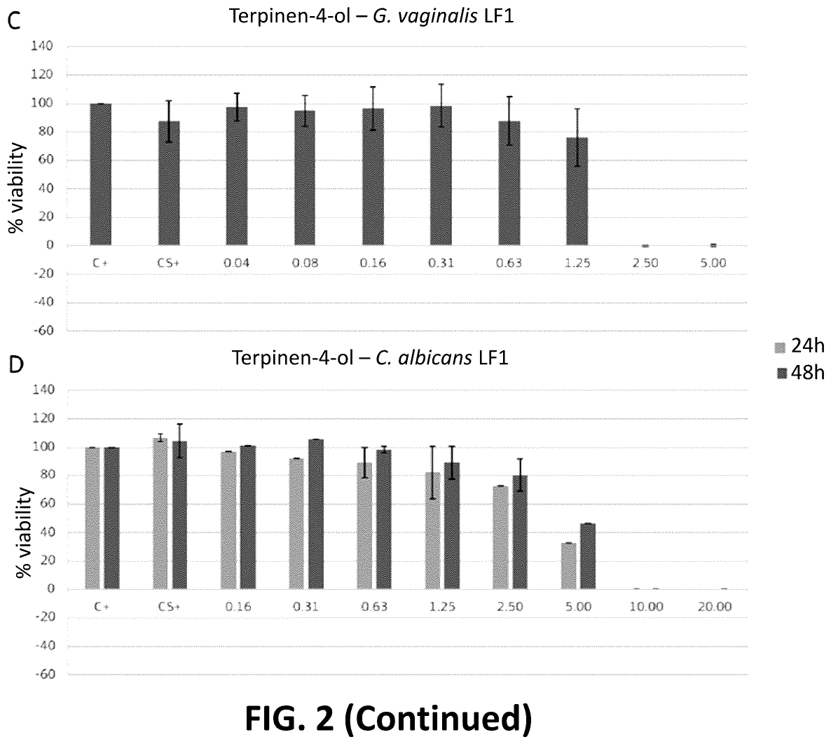

FIG. 2 shows that the MIC of Terpinen-4-ol for Gardenella vaginalis LF1 was 2.5 μl/ml, and that for C. albicans LF1 is 5 μl/ml. The two Lactobacillus strains remained unaffected at either of those doses.

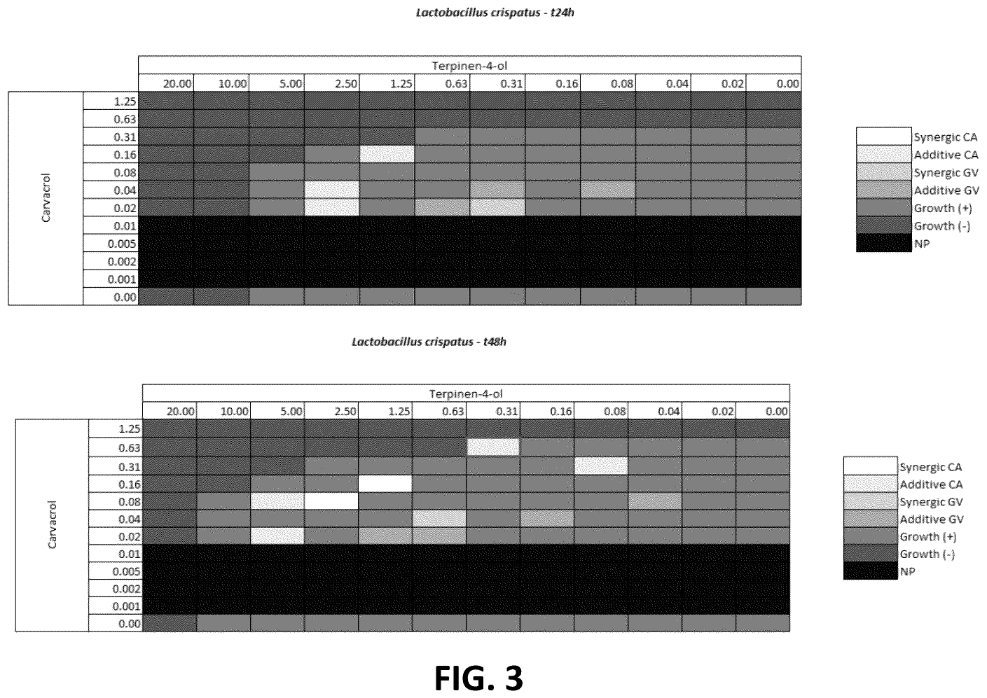

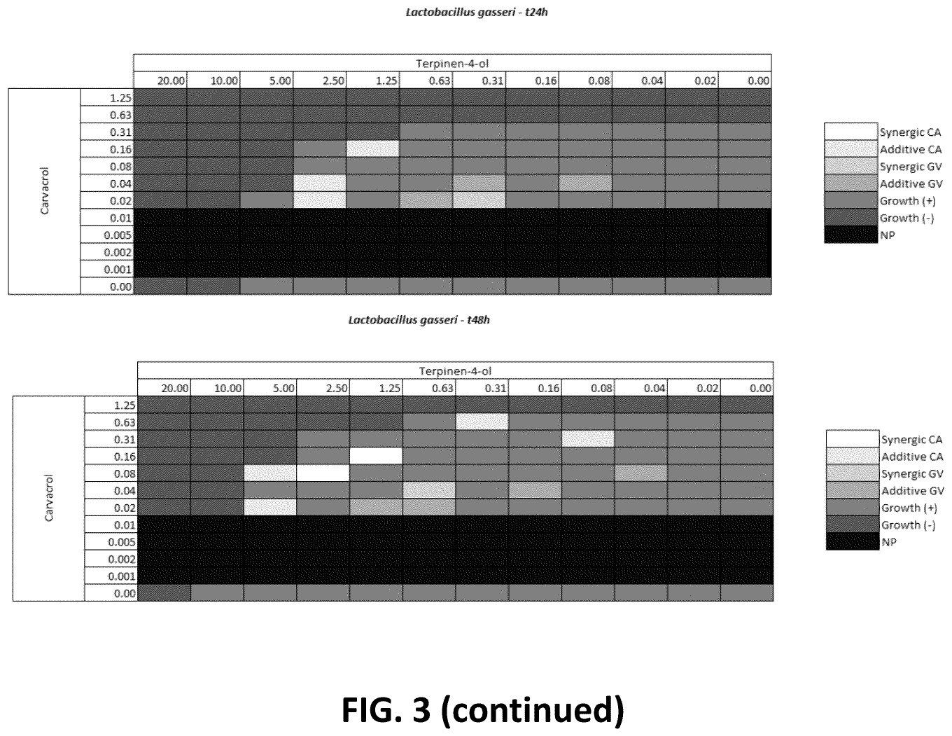

FIG. 3 shows that a dosage of 0.02 μl/ml of carvacrol and 2.5 μl/ml of terpinene-4-ol together shows additive effect to inhibit Candida albicans LF1, and a dosage of 0.02 μl/ml of carvacrol and 0.63 μl/ml of terpinene-4-ol together shows additive effect to inhibit Gardenella vaginalis LF1, while Lactobacillus growth was not inhibited in all those dosages of the EO ingredients.

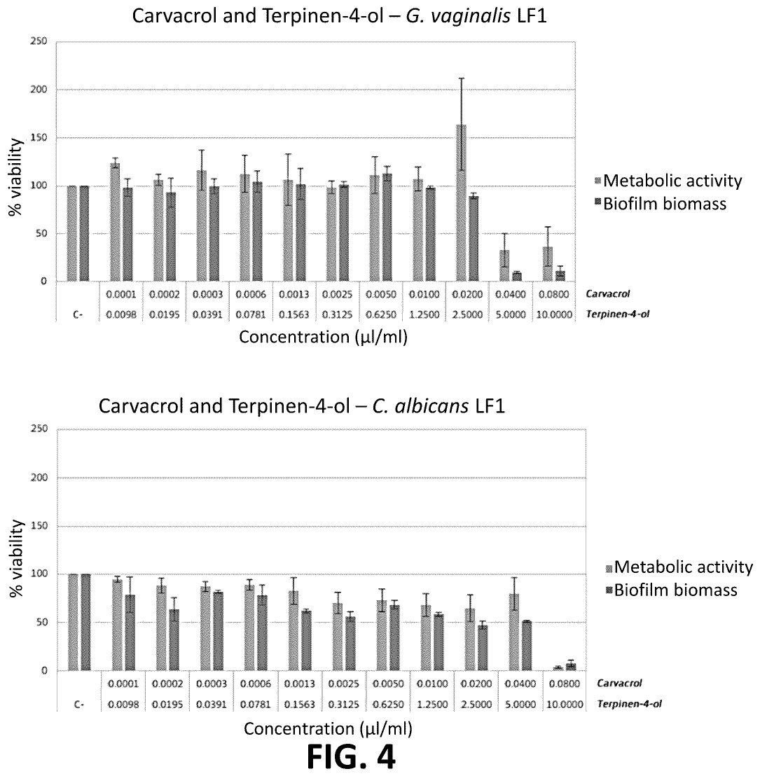

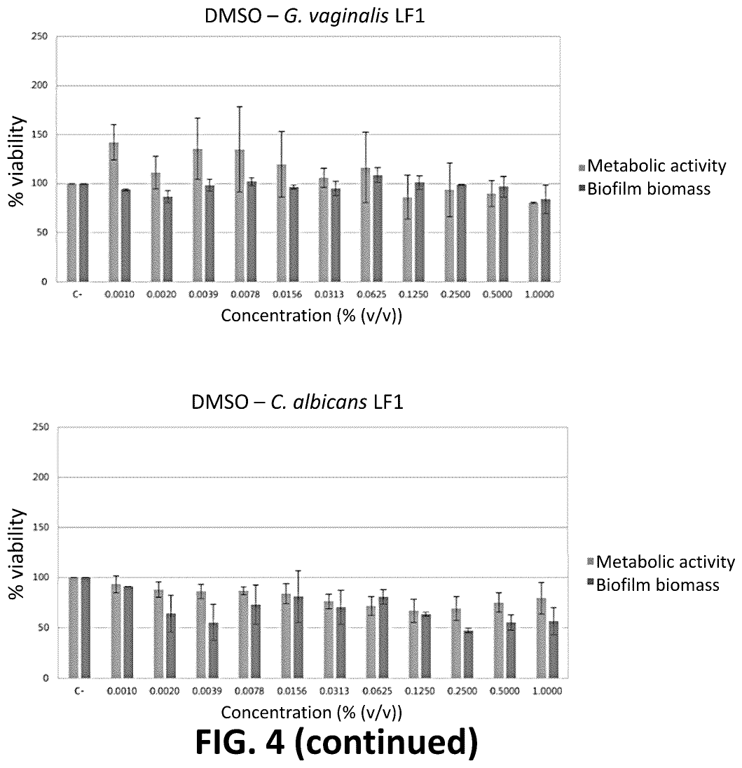

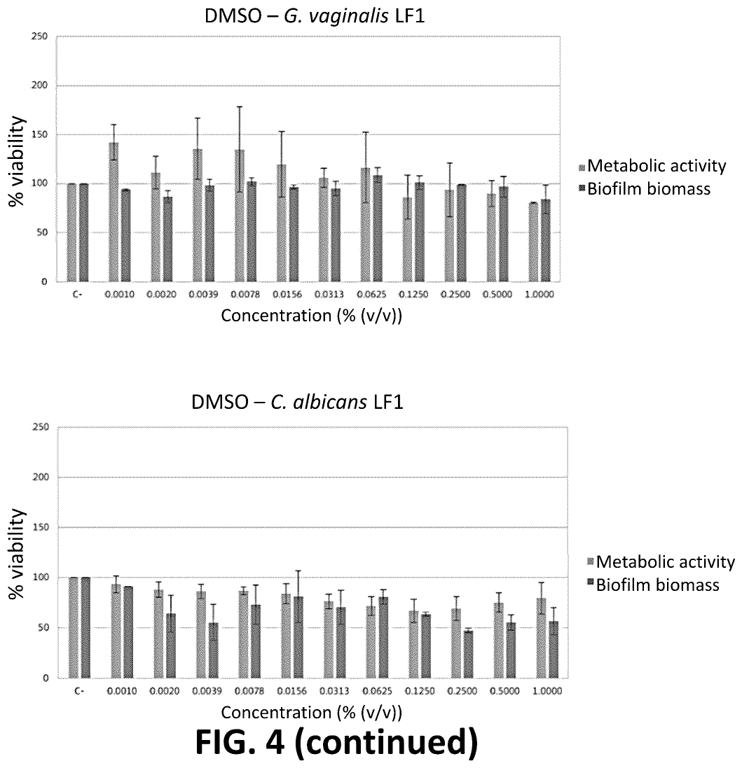

FIG. 4 shows the effect of terpinen-4-ol and carvacrol on the metabolic activity and biofilm biomass of Gardenella vaginalis LF1 and C. albicans LF1 when terpinen-4-ol and carvacrol in different doses were pre-treated to Gardenella vaginalis LF1 and C. albicans LF1 before letting to grow into a biofilm.

Based on the results, carvacrol and terpinen-4-ol, in a double dose response, demonstrated efficacy in inhibiting biofilm formation of G. vaginalis LF1 and C. albicans LF1. The solvent control study showed that DMSO did not influence the determination of metabolic activity in either microorganism or the quantification of biofilm biomass for G. vaginalis. Although, DMSO inhibited about 50% of the biofilm biomass of C. albicans at the first three concentrations tested.

For the microorganism G. vaginalis, biofilm formation was inhibited in the combinations 10 μl/mL terpinen-4-ol/0.08 μl/mL carvacrol and 5 μl/mL terpinen-4-ol/0.04 μl/mL carvacrol. The combination 10 μl/mL terpinen-4-ol/0.08 μl/mL carvacrol inhibited biofilm metabolic activity by 63.45%±20.50% and biofilm biomass 88.86%±5.07%. The combination 5 μl/mL terpinen-4-ol/0.04 μl/mL carvacrol inhibited metabolic activity by 67.29%±17.39% and biofilm biomass by 90.70%±0.88%. The effective concentration that reduced 50% (EC50) of the biofilm metabolic activity corresponds to 4.7824 μl/mL terpinen-4-ol/0.0383 μl/mL carvacrol and for biofilm biomass corresponds to 5.2169 μl/mL terpinen-4-ol/0.0417 μl/mL carvacrol.

Regarding the microorganism C. albicans, the study substances in double response dose showed efficacy in the combination of 10 μl/mL terpinen-4-ol/0.08 μl/mL carvacrol inhibiting the metabolic activity by 96.01%±1.07% and the biomass by 92.08%±3.33%. The inhibition observed in the biofilm biomass is the result of the inhibition caused by the action of the compounds and DMSO, suggesting a synergistic effect between them. Taking into account the inhibition caused by DMSO, the compounds reduce the biofilm biomass by 35.63%±3.33%. The effective concentration that reduced 50% (EC50) of the biofilm metabolic activity corresponds to 2.7052 μl/ml/0.0216 μl/ml terpinen-4-ol and carvacrol, respectively.

It should be appreciated that the present disclosure is not limited to the specific embodiments described above, but includes variations, modifications and equivalent embodiments defined by the following claims.

1. A topical composition for balancing bacteria populations comprising a synergistic effect of:
Carvacrol in an amount of from about 0.002% w/w to about 0.008% w/w; and
Terpinen-4-ol in an amount of from about 0.25% w/w to about 1.0% w/w.

2-3. (canceled)

4. The topical composition of claim 1, wherein the topical composition has Carvacrol and Terpinen-4-ol in a percent ratio of about 2:62.5 to about 4:2000.

5-15. (canceled)

16. The topical composition of claim 1, wherein the topical composition does not contain one or more of added thymol, quercetin, p-cymene, thymoquinone, phenol, eugenol, limonene, aldehyde, parabens, cannabinoids, metal ions, probiotics, xylose, salvia extract, and combinations thereof.

17-36. (canceled)

37. The topical composition of claim 1, wherein the topical composition is for bacterial balance of mucosa, nasopharynx, skin, at least one intimate area, and combinations thereof.

38-48. (canceled)

49. The topical composition of claim 1 wherein the bacterial balance is not of the gastrointestinal mucosa and/or pulmonary mucosa.

50. (canceled)

51. The topical composition of claim 1, wherein the topical composition further comprises at least one ingredient selected from the group consisting of polymer, odor controlling or odor neutralizing agent, surfactant, alcohol, humectant, emollient, buffering agent, pH modifying agent, non-irritating viscosity-increasing agent, contraceptive agent, and combinations thereof.

52-76. (canceled)

77. The topical composition of claim 1, wherein the topical composition is sterile and/or preservative-free.

78-82. (canceled)

83. The topical composition of claim 1, wherein the topical composition is applied in a form selected from the group consisting of a caplet, capsule, cream, drop, film, foam, foaming wash, gel, jelly, liquid, lube, oil, ointment, paste, powder, semi-solid, shampoo, spray, suppository, tablet, wash, and combinations thereof.

84-103. (canceled)

104. The topical composition of claim 1, wherein the topical composition performs one or more of: maintains a healthy bacterial balance, controls odor, prevents discharge, prevents pain, discomfort, or pain and discomfort, prevents or makes the user less susceptible to infection, prevents biofilm formation or maturation, enhances fertility, decreases fertility, and combinations thereof.

105-151. (canceled)

152. The topical composition of claim 1, wherein the topical composition helps to restore or maintain a healthy bacterial balance where there is potential for disruption of a healthy bacterial balance.

153. The topical composition of claim 1, wherein the topical composition helps to restore or maintain a healthy bacterial balance where there is a hormone imbalance.

154. The topical composition of claim 153, wherein the the hormone imbalance is selected from the group consisting of ovulation, menstruation, perimenopause, menopause, and post-menopause.

155-158. (canceled)

159. The topical composition of claim 1, wherein the topical composition helps to restore or maintain a healthy bacterial balance where there is potential for disruption of the healthy bacterial balance caused by a side effect from medication.

160. The topical composition of claim 159, wherein the medication is an antibiotic.

161. (canceled)

162. A method for treating and/or preventing skin or mucosal conditions resulting from microbial overgrowth, imbalance or infections, the method comprising:
administering the topical composition of claim 1 to a subject in need of such treatment or prevention.

163. The method according to claim 162, wherein the microbial agent is at least one bacteria, fungus, or virus.

164-171. (canceled)

172. The topical composition of claim 37, wherein the topical composition is for bacterial balance of nasal mucosa.

173. The topical composition of claim 37, wherein the at least one intimate area is a female intimate area selected from the group consisting of vagina, vulva, perineum, external genitalia, external cervix, and combinations thereof.
